(19) United States
(12) Patent Application Publication
(10) Pub. No.: US 2014/0235504 A1
(43) Pub. Date: Aug. 21, 2014

(54) ANALYTIC METHOD AND DEVICE WITH A MOVABLE PLUNGER

(71) Applicant: ASPRE AG, Appenzell (CH)

(72) Inventor: Friedrich-Josef Sacher, Troisdorf (DE)

(73) Assignee: ASPRE AG, Appenzell (CH)

(21) Appl. No.: 14/347,678

(22) PCT Filed: Sep. 25, 2012
(86) PCT No.: PCT/EP2012/068853

§ 371 Date: Mar. 27, 2014

(30) Foreign Application Priority Data
Sep. 27, 2011 (DE) 10 2011 083 555.5

(51) Int. Cl. C12Q 1/68

(52) U.S. Cl. CPC C12Q 1/686
USPC 506/12; 435/6.12; 435/287.2; 506/39

(57) ABSTRACT

A method and a device for analyzing a liquid is provided, in which the liquid is distributed to a plurality of containers, and which enables the rapid and inexpensive performing of a multitude of analyses in liquids, in particular DNA analyses, in micro-arrays. The liquid is supplied to a transparent analytical plate which includes a matrix with a plurality of at least unilaterally closed receptacle chambers arranged adjacent to each other. Axially-displaceable plungers are moved towards walls of the receptacle chambers and the analytical plates are transported towards the plungers using a transport apparatus. The liquid can be distributed into the receptacle chambers using mobile elements of the analytical plate, and the elements may be moved by the plungers. At least one axially displaceable plunger may be heated to a preset temperature value and impart this temperature to the accommodating chamber.

This description relates to a method and a device for analyzing a liquid, in which the liquid is split between a plurality of containers.

In particular, the description relates to a novel method and a device for carrying out genetic analysis. However, it can also be applied in conjunction with any other analytic methods, in which a liquid to be examined is split between a plurality of containers in order to be checked in respect of specific features after particular chemical, thermal or other treatment steps.

In the practical field of use of genetic analysis, use is made of the polymerase chain reaction (PCR). This is a method in which the genome material DNA is multiplied in vitro. For this purpose, use is made of an enzyme (DNA polymerase) which, in a living being, serves to duplicate DNA. Here, the liquid is heated to a temperature of up to 96° C., generally over a plurality of cycles. For this method, use is made in particular of thermostable DNA polymerases, which maintain their polymerase activity, even at temperatures of almost 100° C.

A plurality of components are required for carrying out a polymerase chain reaction, for example:
the original DNA, which contains the DNA section to be multiplied;
primers which respectively set a start point of the DNA synthesis on the individual strands of DNA, as a result of which the region to be multiplied is delimited from both sides;
thermostable DNA polymerase, which replicates the set section (e.g. Taq-polymerase);
deoxynucleoside triphosphates, from which the DNA strand synthesized by the DNA polymerase is formed;
Mg²⁺-ions;
buffer solutions which create a suitable environment for the DNA polymerase.

A so-called thermocycler is used to carry out the polymerase chain reaction. A thermocycler applies the different temperatures required in the respective steps of the polymerase chain reaction to the liquid, situated in a reaction vessel, with the DNA strand to be replicated. By way of example, the liquid is heated in various steps to temperatures of 60° C., 75° C. and 96° C.

Analytic methods using the polymerase chain reaction are comprehensively described both in the patent literature and the specialist genetic literature. An example of a method for amplifying and sequencing DNA molecules can be gathered from the European patent document EP 0 849 364 B1. A thermocycler and sample container for fast DNA amplification is disclosed in e.g. WO 2009/105499 A1.

In particular, real-time quantitative PCR has proven its worth for quantifying the DNA obtained. The quantification is carried out with the aid of fluorescence measurements, i.e. light measurements, which are detected during a PCR cycle. The fluorescence increases proportionately with the amount of the PCR products. At the end of the plurality of cycles, quantification on the basis of the fluorescence signals is carried out during the exponential phase of the PCR. Correct quantification is only possible in the exponential phase of PCR (which only occurs for a few cycles during the sequence) because ideal reaction conditions are prevalent during this phase. Therefore, this process differs from other quantitative PCR methods (qPCR), which only undertake quantitative evaluation (e.g. competitive PCR) after the PCR has been completed, usually using a gel electrophoretic separation of the PCR fragments.

By way of example, document US 2006/0094027 describes a system for analyzing liquids in so-called micro-arrays. Micro-arrays are fields (arrays) or matrices, which have a multiplicity of very small, discrete amounts of liquid, in the nanoliter range, in a small area. In US 2006/0094027, the micro-arrays are formed by through holes with different hydrophilic/hydrophobic surface properties. The document describes different uses of micro-arrays. Methods for producing micro-arrays on, inter alia, glass surfaces emerge from the documents U.S. Pat. No. 5,807,522 and US 2010/024993 A1. In general, the micro-arrays are formed by virtue of through holes being burnt by laser beams.

It is an object to develop a method and a device which renders it possible to carry out a plurality of analyses in liquids, in particular DNA analyses, in micro-arrays in a quick and cost-effective manner.

With respect to the method, this object is achieved by the totality of features in patent claim 1.

A method is proposed for analyzing a liquid, in which the liquid is split between a plurality of containers, wherein the liquid is fed to a transparent analysis plate which has a matrix with a plurality of receptacle chambers, which are arranged adjacent to one another and closed on at least one side, wherein the liquid is split between the receptacle chambers by means of movable elements of the analysis plate.

Here, use can be made of a novel manufacturing method for treating glass and sapphire, which was developed by the Fraunhofer-Institut für Lasertechnik ILT in Aachen, Germany and is referred to as in-volume selective laser etching (ISLE). In this method, ultrashort pulsed laser radiation is focused within a transparent workpiece and only absorbed in the focus volume. In this focus volume, the optical and chemical properties of the transparent material are modified, without cracks occurring, in such a way that it becomes possible to chemically etch it in a selective manner. By moving the focus with the aid of a micro-scanner, those regions which are intended to subsequently be removed by wet chemical etching are exposed. This renders it possible to produce micro-channels, shaped bores, structured components and even complex assembled mechanical systems in glass. More detailed information relating to selective laser etching can be gathered from the publication in the magazine “Mikroproduktion 06/2010”, published by Carl Hanser Verlag, Munich, pages 10-13, ISSN 1614-4538. Aspects of this manufacturing technology can be gathered from the article by Maren Hörstmann-Jungemann, Jens Gottmann and Dirk Wortmann, “Nano- and Microstructuring of SiO₂ and Sapphire with fs-laser Induced Selective Etching” in JLMN—Journal of Laser Micro/Nanoengineering, volume 4, number 2, 2009, pages 135-140. The production of micro-fluid channels is described by Osellame et al. in the journal “Laser Photonics Review” 5, number 3, pages 442-463 (2011).

The structures of the novel device for carrying out the novel method are preferably separated out of transparent plates by selective laser etching. In particular, this method renders it possible to provide receptacle chambers to a transparent analysis plate closed on at least one side, wherein at least one movable element of the analysis plate fills the liquid into the receptacle chamber. As a result of the fact that the receptacle chambers are closed on at least one side, the risk of the liquid emerging from the receptacle chambers during the analysis procedure is avoided. This guarantees a promising analysis result. This also reduces the risk of contaminating the surroundings with the liquid to be analyzed, which is very advantageous, particularly when examining poisonous or dangerous substances. The movable element can bring about a change in volume in the receptacle chamber or a volume connected therewith. This causes the receptacle chamber to be filled with liquid. The use of movable elements renders possible the reliable supply of precise amounts of liquids, which can be very small, e.g. a few nanoliters or milliliters.

It is possible to etch a transparent plate with through holes, which are sealed, at least on one side, by a cover plate or any other cover layer. Selective laser etching can also be carried out in the case of a transparent plastic, and so the analysis plate can alternatively be produced from a plastic plate, into which the through holes are etched.

However, the analysis plate can also be formed alternatively, for example cast from plastic.

A transportation device can be used to transport the analysis plate to plungers, which can be displaced axially against walls of the receptacle chambers. As a result of this, the analysis plate itself does not require complicated drive means or conveying means for conveying the liquid. Each of the plungers can be coupled to a micro linear drive, which moves the former. Alternatively, the plungers can be coupled to drives in groups. The plate does not have any drive elements and can be manufactured with little outlay and at low costs. It can be manufactured as a mass-produced article. The more complicated transportation devices and plunger arrangements with their drives do not come into contact with the liquid to be analyzed. Therefore, they can be used very many times in conjunction with the cost-effective analysis plates.

In the form of a summary, the following description describes all development aspects of the novel analytic method according to the current state of development.

It should be noted that all features and partial features of the following description with respect to the novel method and the novel device may be advantageous. Here, the features, partial features and functions explained in the following description can be implemented on their own and can be combined with one another in any expedient manner. In particular, features, partial features and functions described in conjunction with another feature or a specific embodiment are not coupled to the specified other feature or the specified embodiment. All features, partial features and functions of the following description are independently suitable for developing the development described herein in an advantageous manner.

A matrix of a plurality of receptacle chambers arranged next to one another is etched out of a solid glass or plastic plate with a thickness of e.g. 1 to 8 mm in such a way that said receptacle chambers are closed or sealable on at least one side. By way of example, it is possible to produce hexagonal receptacle chambers in the analysis plate in the style of a honeycomb. The distance between two parallel edges of a hexagonal receptacle chamber can lie in the region of 1 mm.

This renders it possible to form receptacle chambers (which are occasionally also referred to as reactor chambers) with a very small volume. Thus, the volume of a single chamber can be much less than 1 μl.

Since the analysis plate with the receptacle chambers itself is transparent, it is possible initially to treat and subsequently analyze the liquid contained therein without transferring it within the reactor chamber thereof. In particular, light radiation can be recorded through the transparent material of the plate, which light radiation emanates from the liquid in the receptacle chamber. In particular, this applies to fluorescence measurements within the scope of real-time quantitative PCR. The fluorescent substances in the receptacle chamber can be irradiated through the transparent analysis plate with light, just like the light emitted by the fluorescent substances can be guided through a ply of the transparent plate.

A very efficient analytic method is developed by using a transparent analysis plate with a matrix of receptacle chambers. The analysis plate with the receptacle chambers can be supplied to different treatment and analysis stations, wherein the liquid to be analyzed is enclosed in the receptacle chamber. Applying the temperature to the liquid in each receptacle chamber is brought about in this case through the wall of the receptacle chamber. By way of example, the stamps or plungers assigned to the individual receptacle chambers can be used to this end.

The liquid in the receptacle chamber cannot leak from the analysis plate. Consequently, merely the analysis plate needs to be disposed of as hazardous waste. The equipment is not contaminated by the liquid. There is no risk of releasing e.g. dangerous viruses contained in the liquid. Therefore, a device for analyzing the liquid in the receptacle chambers of the transparent analysis plate can be successively charged with any number of analysis plates which contain liquid in the receptacle chambers thereof.

In practice, the analysis plate can have flow channels which open into the receptacle chambers. These flow channels can also be worked out of the analysis plate, in particular by selective laser etching. The flow channels can connect the receptacle chambers to one another or to filling devices.

The receptacle chambers and/or the flow channels are sealed on at least one side such that the analysis plate has a cover layer which is continuous and transparent. The transparent cover layer seals the receptacle chambers and/or flow channels on this side and thus avoids leakage of liquid from the receptacle chambers or the flow channels. It can have an at least partly transparent configuration so that light radiation can pass therethrough.

The receptacle chambers and/or the flow channels can be sealed by movable pistons which form the movable elements. By moving the pistons, it is possible to change the volume of the receptacle chamber and/or the flow channels. The volume changes are of great importance, in particular when filling the receptacle chambers. This procedure is described in more detail below.

Furthermore, the pistons can be transparent. In particular, the pistons can be formed from the transparent analysis plate using the above-described method of selective laser etching. In the process, the pistons can be etched out of the material of the receptacle plate, wherein material undercuts can keep the pistons in the receptacle plate.

The liquid can be filled into a filling chamber in the transparent analysis plate, from where it is guided into a flow channel which guides the liquid into at least one receptacle chamber. The filling chamber can have an upper opening, which is sealed with a plug after filling in the liquid. The plug need not necessarily be manufactured from the transparent material of the analysis plate. The plug can be fixed in the opening by elastic deformation. Alternatively, the plug can be adhesively bonded in the opening by a binder or an adhesive.

Plungers, which are displaceable in the axial direction thereof, can be moved against a wall of a receptacle chamber and, in particular, against the pistons. In particular, the receptacle chamber can be sealed by a displaceable sealing piston on the side lying opposite to the transparent cover layer. An axially displaceable plunger can be displaceable against the underside of the sealing piston.

In order to move the plunger against the wall of the receptacle chamber, provision can preferably be made for an electronically actuatable micro linear drive, to which the plunger is coupled. In particular, the micro linear drive can have an actuator or a part connected therewith, which runs on a slipway that converts movements of the actuator into vertical lifting or lowering movements. By way of example, such a micro linear drive emerges from the international patent application PCT/EP2011/003090, the content of which is incorporated into the present patent application by reference. The actuator is preferably a piezoelectric actuator, which changes its dimensions by applying a voltage. A wedge-shaped slide on which the plunger is supported can be connected to the actuator. Alternatively, provision can be made for two parts with a coiled slipway, which are rotatable with respect to one another, the movable part of which is coupled to the actuator. By activating the piezoelectric actuator, the movable part moves along the slipway, resulting in lifting or lowering. The plunger connected therewith follows this movement. Such a micro linear drive can be produced with very small dimensions at low costs.

The axially displaceable plunger can be connected to a heating device which heats the axially displaceable plunger to a predefined temperature value. If the micro linear drive is used to move the axially displaceable plunger against the wall of the receptacle chamber, said axially displaceable plunger impresses a predefined temperature onto the receptacle chamber and the liquid contained therein. In this manner, the temperature cycles required for the polymerase chain reaction are produced within the receptacle chambers. Furthermore, provision can be made for cooling devices for cooling the receptacle chambers in order to expose the receptacle chambers to a defined temperature cycle. In particular, cooling channels, through which cooling air is guided, can be provided in the components of the analysis plate adjoining the receptacle chamber.

In a manner similar to the axially displaceable sealing piston, an axially displaceable separating piston can be used in the region of a flow channel, which separating piston can be moved against the ceiling of the flow channel in order to drive the liquid film out of the flow channel and separate it. Initially, the liquid is driven out of the filling chamber, preferably also by an axially displaceable piston, and displaced into a first receptacle chamber and into downstream flow channels and receptacle chambers through a first flow channel. After the filling chamber has been completely emptied, an axially displaceable separating piston can be pressed against the ceiling of the flow channel adjoining the filling chamber. The separating piston presses the liquid completely out of the flow channel. The receptacle chamber lying next to the flow channel is filled with liquid. The receptacle chamber is adjoined by a second flow channel containing a certain amount of the liquid. By displacing a further separating piston against the ceiling of this second flow channel, the first receptacle chamber is sealed on the second side and a fixedly defined liquid volume is enclosed in this receptacle chamber. The liquid volume can be very small and can lie in the region of less than 1 μl (e.g. 0.4 μl) or a few nl (nanoliter). The separating piston can be displaceable in a piston chamber below a flow channel, wherein separating piston and piston chamber have complementary latches and latch receptacles, which latch into one another when the separating piston lies against the ceiling of the flow channel. This is how the separating piston is locked in the position resting against the ceiling of the flow channel. The latches and latch receptacles can likewise be worked out of the material of the analysis plate by selective laser etching. The flow channel is formed by the region of the piston chamber lying above the separating piston. This latching device is advantageous, but not mandatory. If the separating piston and the piston chamber, in which the separating piston is held, are manufactured with sufficiently small tolerances, the separating piston pressed against the ceiling of the flow channel by a plunger adheres to the ceiling and is not detached therefrom again purely as a result of the gravitational force.

In practice e.g. eight receptacle chambers can adjoin each filling chamber, wherein each filling chamber is connected to the first receptacle chamber via a first flow channel. The receptacle chambers are in each case connected to the subsequent receptacle chambers by a flow channel. Each flow channel can be closed off by a lockable separating piston. After all separating pistons have been successively pushed against the ceiling of the associated flow channel, starting from the flow channel adjoining the filling chamber, the aforementioned small liquid volume is contained in each of the receptacle chambers. The liquid can be subjected to the temperature cycles required for the analysis by impressing the predefined temperatures onto the receptacle chambers. Subsequently, the liquid in the transparent analysis plate can be analyzed optically. The optical analytic method is described further below.

An edge can be provided on the top side of the separating piston, which edge is pressed against the ceiling of the flow channel in order to separate the liquid film in the flow channel. The edge can also be formed from the glass material of the analysis plate by selective laser etching. The edge can be displaceably arranged in the separating piston and inserted into the separating piston when pushing the separating piston against the ceiling of the flow channel. The edge separates the liquid film in the flow channel at a predefined position. Due to the fact that the flow channel has a very thin cross section (1 mm and less), surface forces play a significant role in filling the receptacle chambers. The edge on the top side of the separating piston ensures that the liquid film is reliably separated in the flow channel and two separate liquid amounts form on either side of the edge.

The receptacle chambers can have a hydrophilic surface, which is well wetted by water, at least in the region of the surface of the sealing piston and the ceiling of the receptacle chamber lying opposite this surface. Plane glass surfaces are generally hydrophilic. Selective laser etching renders it possible to produce very smooth surfaces, to which water adheres very well. Additionally or alternatively, the surfaces can be made smooth by laser action in order to have ideal hydrophilic properties. In the process, laser etching can initially produce rough, hydrophobic surfaces which are irradiated by laser beams in a second step during which unevenness is ablated in order to become hydrophilic.

By contrast, at least the sidewalls of the receptacle chambers and/or the walls of the flow channels can be provided with a hydrophobic surface. By way of example, hydrophobic surfaces are produced by a high surface roughness. Such a surface roughness can also be realized by selective laser etching. It is alternatively or additionally possible for the hydrophobic surfaces to be coated by a hydrophobic substance such as PTFE. As a result of the sidewalls of the receptacle chambers and/or the walls of the flow channels, including the top sides of the sealing pistons delimiting the flow channels to the bottom, having a hydrophobic, i.e. water-repelling configuration, the small liquid volume (less than 1 μl) forms a drop within the receptacle chamber, which drop adheres to the top side of the receptacle chamber and the surface of the sealing piston, but is repelled from the remaining walls of the receptacle chamber, and which drop has strong cohesion within the receptacle chamber due to surface tension. As a result of this, the liquid volume is reliably enclosed within the receptacle chamber and the sealing piston fixes with close contact to liquid within the receptacle chamber. The displaceable sealing pistons can even have air channels on their outer side. This allows air to flow out during the filling procedure of the receptacle chambers. Since the liquid adheres to the hydrophilic surface of the receptacle piston, said piston is fixed in its position delimiting the liquid volume. Furthermore, as a result of the surface tension there is no risk of liquid leaking through the air channels that have a diameter of a few micrometers. The surface of the sealing piston can also have a hydrophobic configuration in the region of the air channels in order to counteract penetration of liquid from the receptacle chamber.

Reagents, in particular enzymes, markers and the further components required to carry out a polymerase chain reaction can be filled into the receptacle chambers and/or the filling chambers. For this purpose, reservoirs assigned to the receptacle chambers and/or the filling chambers can be provided in the analysis plate, which reservoirs have a fluid connection to the receptacle chambers and/or filling chambers. By way of example, a channel can lead from the reservoir to the filling or receptacle chamber. Ejection pistons, which delimit the volume with the reagents, can be arranged in the reservoirs. By displacing the ejection pistons, the reagents are pressed from the reservoir into the receptacle chamber/filling chamber through the channel. Consequently, the design of the reservoir is similar to the design of the receptacle chamber or the flow channel, wherein the reservoirs can be filled during the production of the analysis plate, whereas the liquid to be analyzed is filled into the receptacle chambers via the filling chambers directly before the analysis. Consequently, the ejection pistons also correspond to the sealing pistons in the receptacle chambers or the separating pistons in the flow channels.

A method for analyzing a liquid, which is split between a plurality of containers, can comprise the following steps:

A) the liquid is fed to a transparent analysis plate, which has a matrix with a plurality of receptacle chambers, which are arranged adjacent to one another and closed on at least one side, wherein the liquid is split between the receptacle chambers,
B) a first temperature is impressed onto each receptacle chamber in the analysis plate through a wall of the receptacle chamber during a first period of time,
C) a second temperature is impressed onto each receptacle chamber in the analysis plate through a wall of the receptacle chamber during a second period of time.

As explained at the outset, the transparent analysis plate forms a matrix with a multiplicity of receptacle chambers, in which the liquid to be analyzed is held, sealed from the surroundings. By impressing different temperatures, the individual receptacle chambers can be exposed to the temperature cycles required for the analysis, in particular for the polymerase chain reaction. The temperature can be impressed by virtue of the fact that a plunger heated to a specific temperature value is brought into contact with a wall of the respective receptacle chamber. In particular, a plunger can, in practice, be pressed against the base wall and impress the temperature onto the latter. After the first period of time, during which the first temperature is impressed, has passed, the analysis plate can be transported to a different position, in which the receptacle chamber is contacted by a second plunger at a second temperature, by a transportation device. The analysis plate can successively be transported in such a way that the receptacle chambers thereof are successively exposed to different temperatures. By way of example, provision can be made for a plunger matrix which corresponds to the matrix of receptacle chambers. The analysis plate is moved forward in each case by the spacing of a plunger row such that the row of receptacle chambers is successively fed to different plunger rows. The analysis plate can also be transported forward and backward such that a specific row of the receptacle chambers is initially fed to a first plunger row, subsequently to a second plunger row, thereafter to a third plunger row and subsequently back to the first plunger row. Each of the plunger rows can be preheated to a specific temperature. In this manner, the receptacle chambers of the analysis plate can successively be exposed to different temperatures.

Furthermore, provision can be made for suitable cooling devices, such as e.g. fans, which cause the temperature in the receptacle chambers to sink after the contact between the receptacle chambers by the plungers is completed. In particular, the plungers can be brought into contact with the receptacle chambers by means of the above-described micro linear drive. In particular, the fans can drive cooling air through cooling channels in the region of the receptacle chambers. Naturally, it is also possible to produce negative pressure on one side of the analysis plate, which negative pressure suctions surrounding air through cooling channels in the analysis plate. Here, the cooling air flows around the plunger and therefore prevents heat transfer as long as there is no physical contact between the plunger and the analysis plate.

As mentioned above, the analytic method is preferably performed by detecting optical signals. To this end, the analysis plate is fed to at least one row of optical sensors, in particular photosensors, such that each optical sensor is spatially assigned to one of the receptacle chambers. The optical sensors in the row are spaced apart by the distance corresponding to that between two receptacle chambers in a row of adjacent receptacle chambers of the analysis plate. In a practical embodiment, the optical sensors are arranged in a plurality of rows such that a matrix of optical sensors which corresponds to the matrix of receptacle chambers in an analysis plate is produced. It is also possible to provide a matrix of photosensors which correspond to part of the matrix of the receptacle chambers, for example half of the receptacle chambers or a third of the receptacle chambers. For analysis purposes, corresponding portions of the analysis plate are then successively supplied to the matrix of optical sensors by means of a transportation device. The optical sensors can be arranged above the receptacle chambers. On its closed side, the analysis plate has the transparent cover layer, wherein the optical sensors record a photographic image of the liquid in the receptacle chambers through the transparent cover layer of the analysis plate.

Furthermore, the analysis plate can be fed to a row of light sources, wherein each light source is in each case spatially assigned to one receptacle chamber. The light source is also preferably arranged above the transparent cover layer of the receptacle chamber. In practice, the light source can be arranged adjacent to the photosensor. Consequently, the light sources are likewise arranged in a matrix, wherein the analysis plate is transported by means of a transportation device into a position in which the whole matrix of receptacle chambers, or at least part of this matrix, is arranged below the photosensors and the adjacent light sources. The receptacle chambers can be illuminated by the light sources. The photosensors record the fluorescent afterglow of the liquid in the receptacle chambers. In particular, the combined photosensor/light source pairs can be arranged in the form of a matrix corresponding to the matrix of receptacle chambers. One pair comprising photosensor and light source is in each case assigned to one of the receptacle chambers. The analysis plate with the receptacle chambers can be brought into a position by means of a transportation device, in which position each receptacle chamber lies directly below a light source and a photosensor.

Light-emitting diodes (LEDs), in particular organic light-emitting diodes (OLEDs), can be used as light sources. By way of example, CMOS image sensors can be used as photosensors.

The signals recorded by each photosensor can, in practice, be supplied to a database. The measurement values for all receptacle chambers of an analysis plate can be stored in this database. This is how data records which contain the analysis results for all receptacle chambers of an analysis plate are produced.

The analysis plate can also be transported to the photosensors and, optionally, the light sources by means of a suitable transportation device after a predefined number of thermal cycles such that a recording is performed after a first number of thermal cycles. After this recording, the analysis plate can be transported back to the device for the thermal treatment and fed back to the photosensors for recording measured values after a further temperature cycle.

Furthermore, a device for analyzing a liquid is described, wherein the device comprises a plurality of containers between which the liquid can be split. The device comprises at least one transparent analysis plate which has a matrix with a plurality of receptacle chambers, which are arranged adjacent to one another and closed on at least one side, wherein the liquid can be split between the receptacle chambers. A flow channel in the analysis plate can be provided between respectively two receptacle chambers. Receptacle chambers and/or flow channels are covered by a continuous transparent cover layer. The analysis plate can furthermore comprise movable elements for sealing the receptacle chambers and/or the flow channels. The movable elements can be displaceable filling pistons, sealing pistons or separating pistons, which are likewise preferably at least partly transparent. The analysis plate with the receptacle chambers and the movable elements can be produced from a transparent plate by selective laser etching. In practice, the transparent plate can consist of glass. However, use can also be made of a transparent plate made of plastic. The analysis plate can have at least one filling chamber, from which a flow channel leads into at least one receptacle chamber. The analysis plate preferably has a plurality of filling chambers following one another at a constant distance on one edge. A row of receptacle chambers can adjoin each filling chamber. Each filling chamber can be used to fill a row of receptacle chambers with the liquid to be analyzed.

The filling chamber can have an upper opening, into which liquid is filled. After filling the liquid into the filling chamber, the upper opening can be sealed with a plug.

The device can furthermore, as described above, have axially displaceable plungers which can be moved against a wall of the receptacle chamber, wherein each plunger can be coupled to an electronically actuatable micro linear drive. The axially displaceable plungers can be heated to a predefined temperature value and transfer this temperature to the liquid in the receptacle chamber via the chamber wall.

The analysis plate of the device can furthermore comprise the above-described separating pistons, latches, latch receptacles and edges. Reagents, in particular enzymes, required for the liquid analytic method and the further components required for the polymerase chain reaction be introduced into the receptacle chambers of the analysis plate. To this end, the analysis plate can have reservoirs, in which the reagents are held and which have a fluid connection to a filling chamber or a receptacle chamber. Ejection pistons can be provided in the reservoirs and these eject reagents from the reservoirs.

The device can furthermore comprise suitable heating devices, which impress predefined temperatures onto the receptacle chambers. In particular, these heating devices can be plungers arranged in rows.

Furthermore, the device can have a transportation device, which feeds the transparent analysis plate to the heating devices in such a way that the heating devices can act on the receptacle chambers of the analysis plate.

Finally, the device can comprise optical sensors (photosensors) and/or light sources, wherein the transportation device can align the analysis plate in relation to the photosensors and/or light sources. In the aligned position, a receptacle chamber preferably lies below a photosensor or below a light source.

Embodiments will be described below with reference to the attached figures.

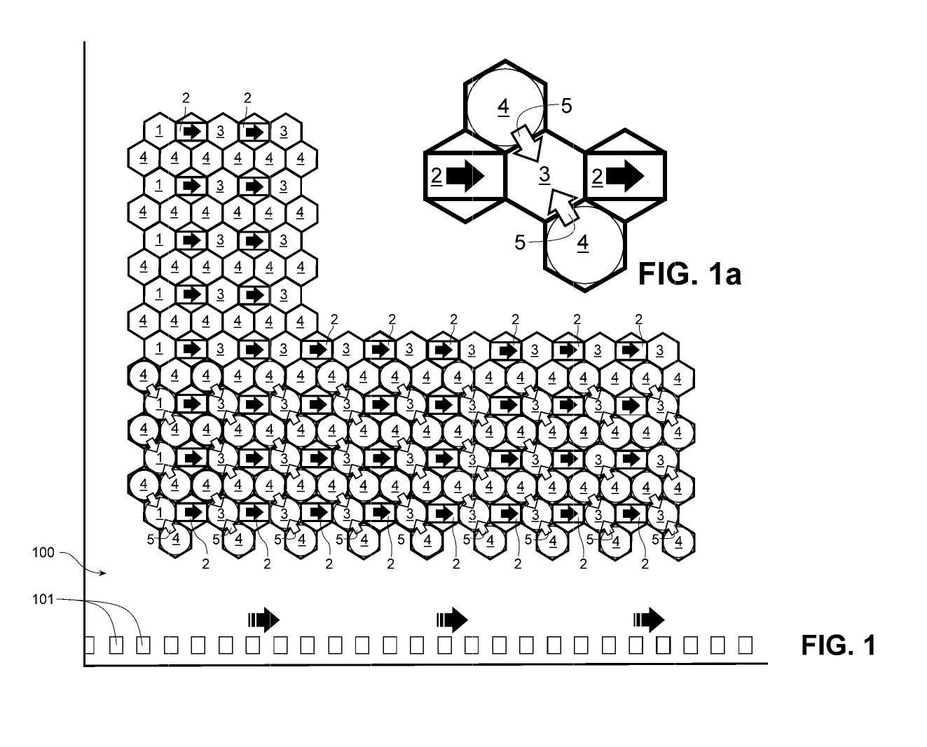

FIG. 1 schematically shows a sectional top view of an analysis device and FIG. 1a shows a magnified cutout of a section of the analysis plate.

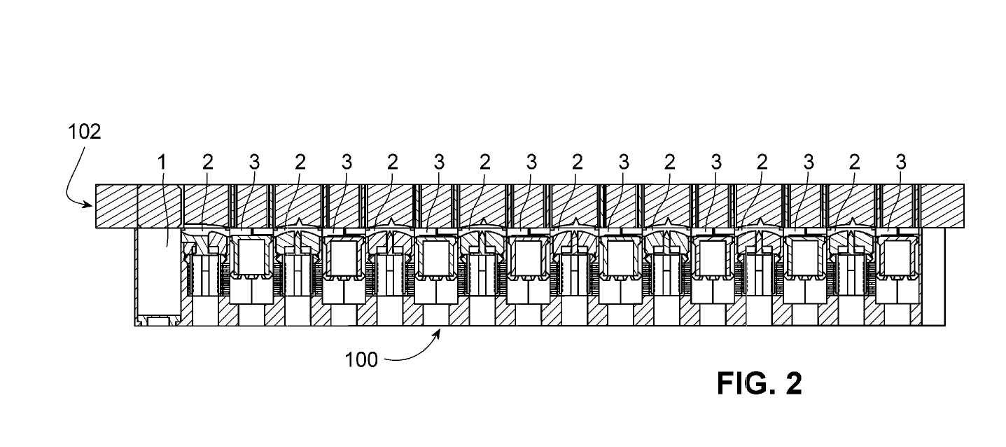

FIG. 2 shows a schematic sectional illustration of the analysis plate from FIG. 1.

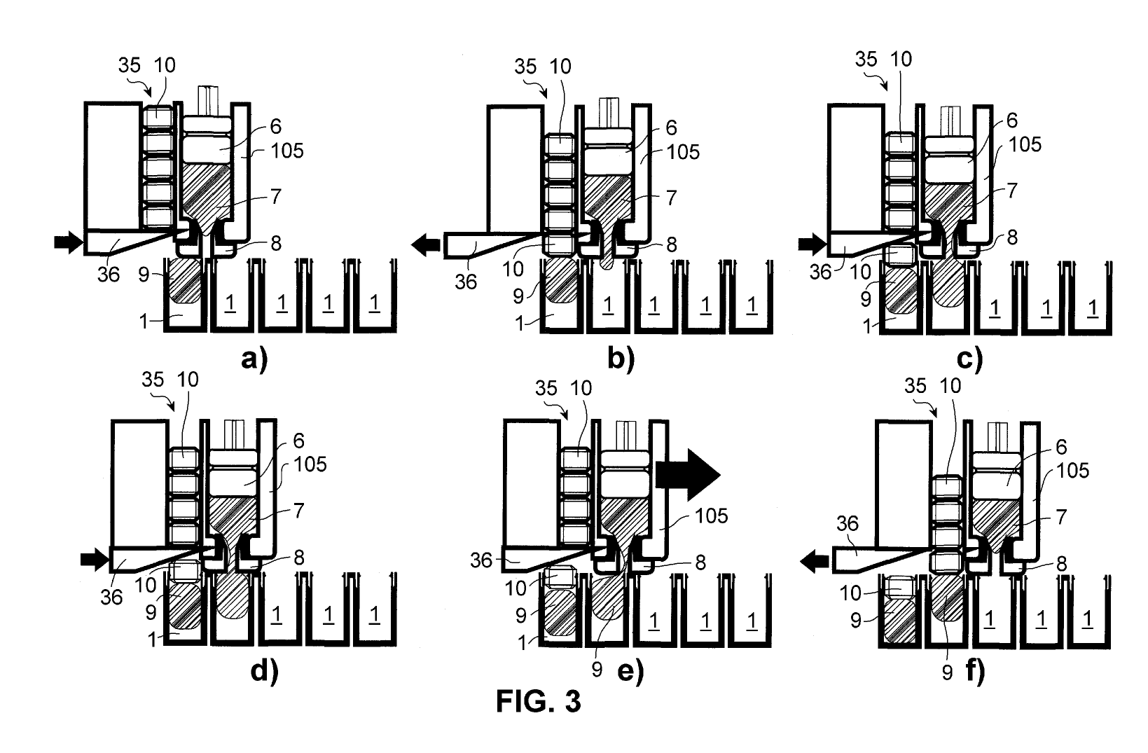

FIG. 3 shows six views of a filling device for the filling chambers of the analysis plate of the analysis device.

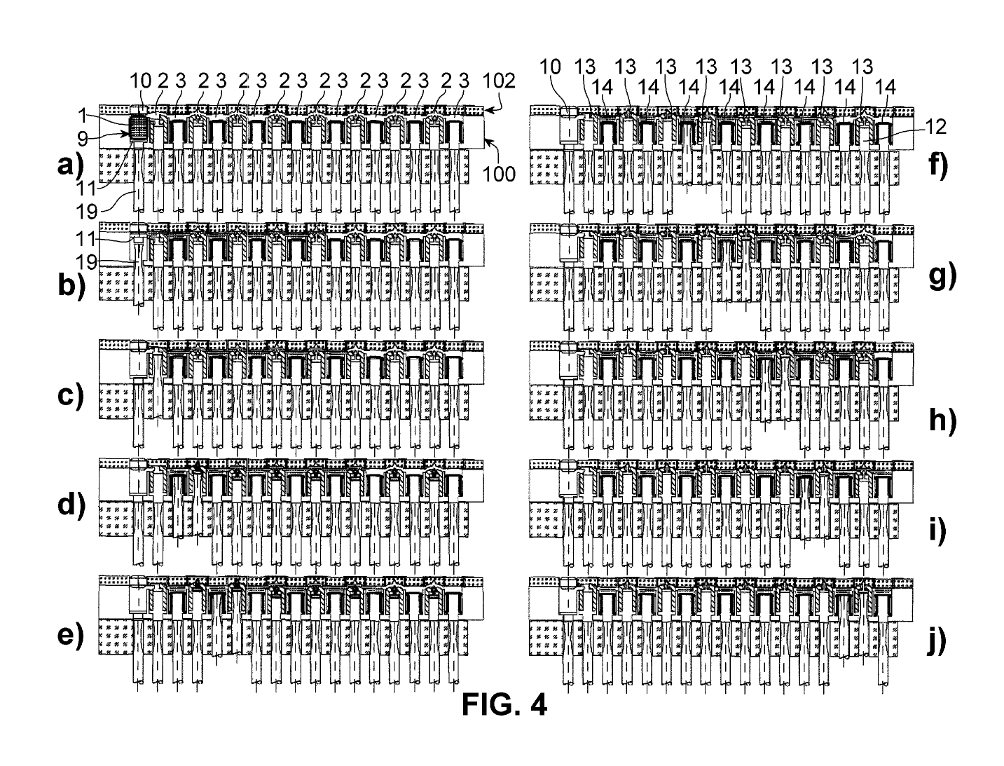

FIG. 4 shows ten views which, in a sectional illustration, explain the filling process of the analysis plate.

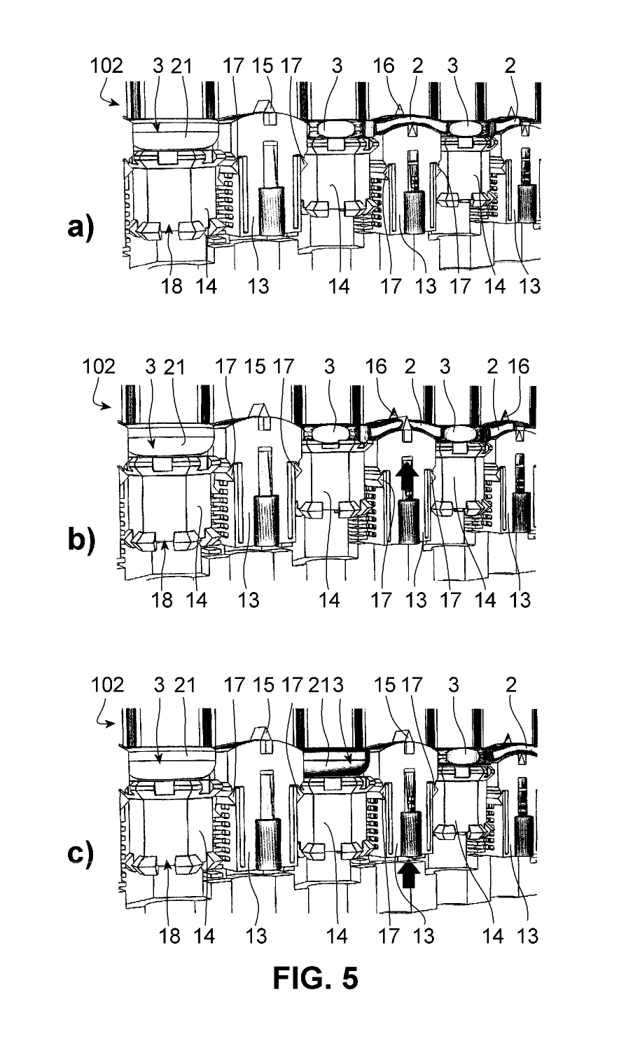

FIG. 5 shows three views of a magnified cutout of the analysis plate during the filling process.

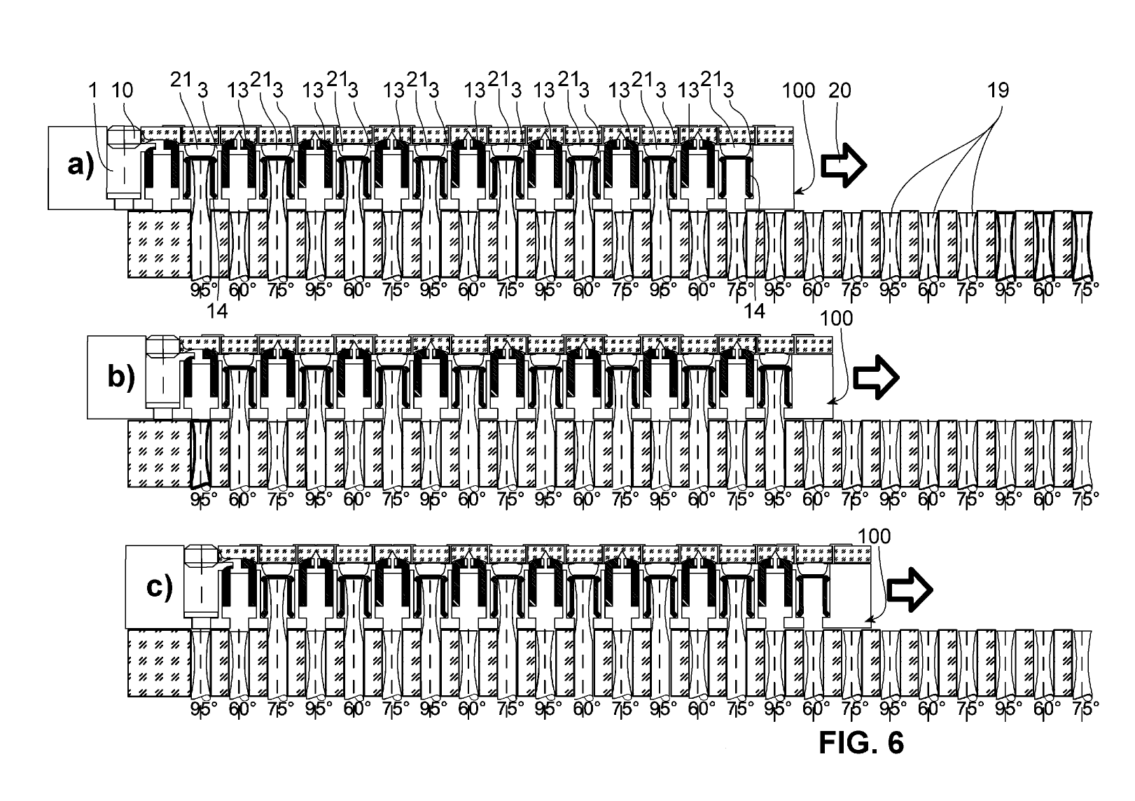

FIG. 6 shows the filled analysis plate while carrying out thermal treatment steps in three illustrations in a sectional schematic view.

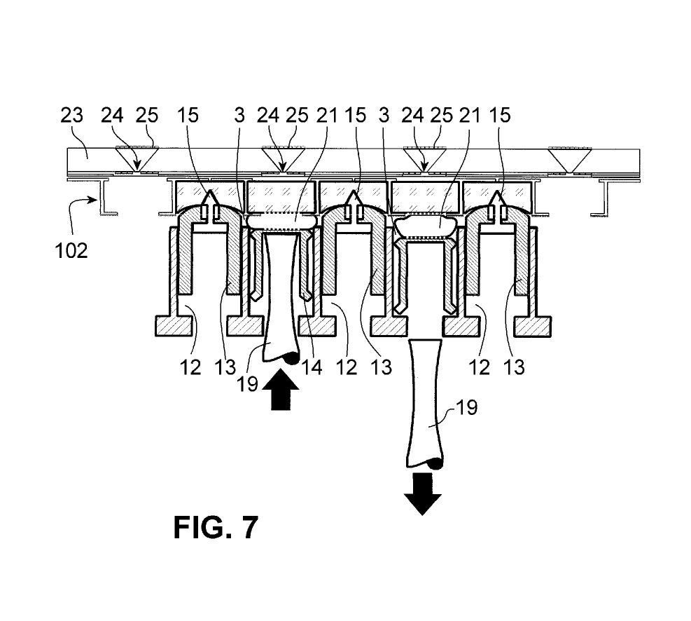

FIG. 7 shows details of the analysis plate during the thermal treatment.

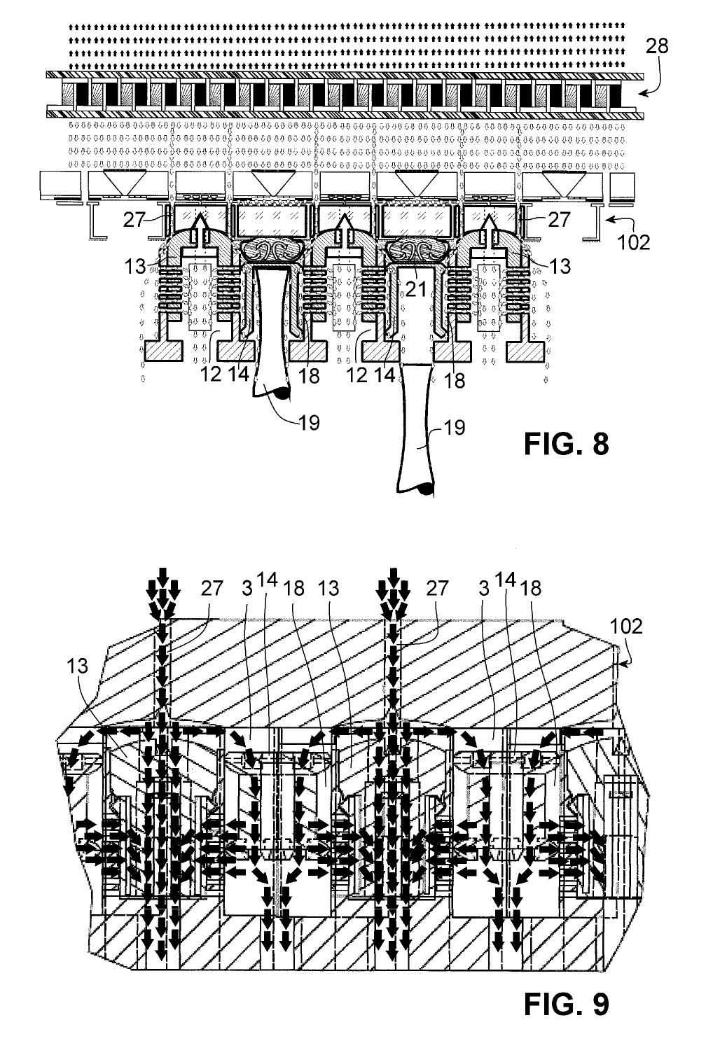

FIG. 8 shows a sectional illustration of the analysis plate, which explains the cooling system of the plate.

FIG. 9 shows a magnified illustration of a cutout from FIG. 8.

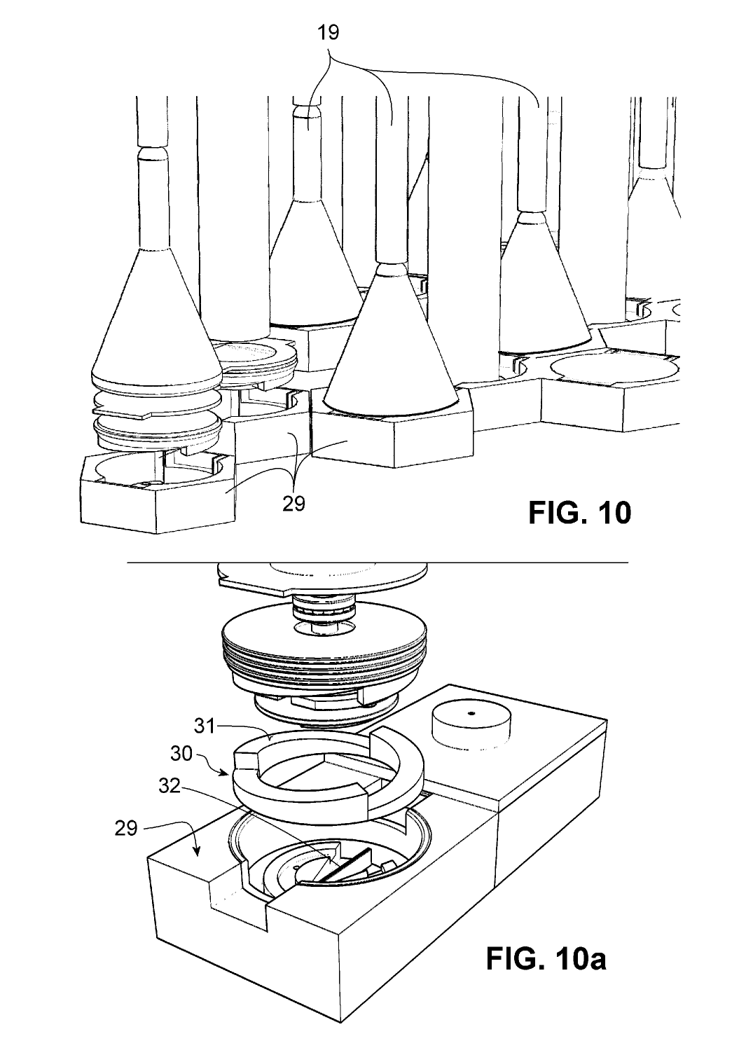

FIG. 10 shows a micro drive for the plungers of the analysis device and FIG. 10a) shows a magnified three-dimensional illustration of the micro drive.

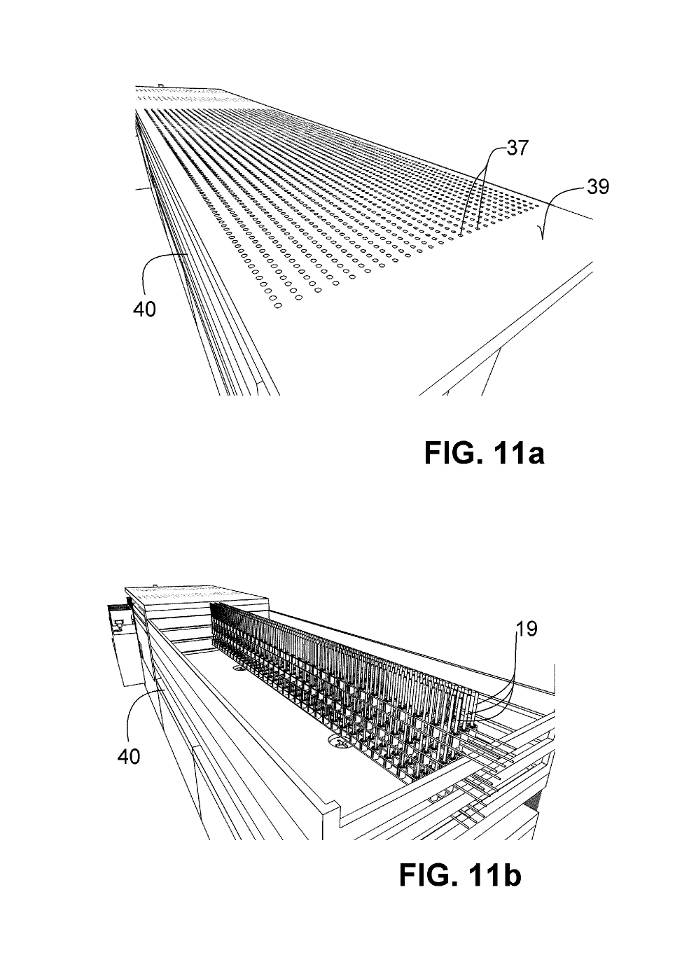

FIG. 11a shows a schematic illustration of a bearing surface for the analysis plate and FIG. 11b shows the matrix of plungers below the bearing surface.

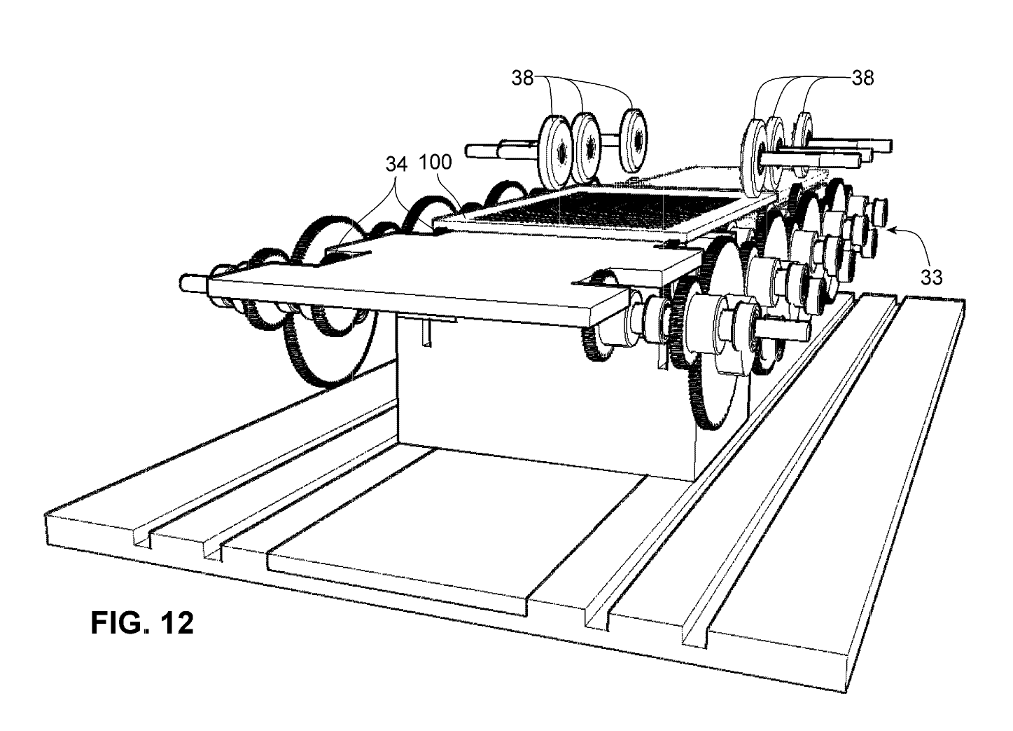

FIG. 12 shows a schematic illustration of a transportation device for the analysis plate of the analysis device.

In FIG. 1, a matrix of the various cavities of an analysis plate 100 for the analysis device can be seen. The basic material of the analysis plate 100 is transparent and preferably glass or else plastic. FIG. 1 shows a top view of the analysis plate 100 without the cover layer applied thereon. Laser etching has etched a honeycomb structure out of the basic material of the analysis plate 100.

Filling chambers 1 are provided in the left-hand region of the analysis plate 100. Each filling chamber 1 consists of a honeycomb of the honeycomb structure of the analysis plate 100. Each filling chamber 1 is connected via fluid connections 5 to two adjacent reservoirs 4, which contain reagents. An ejection piston can be used to selectively fill the reagents from each of the reservoirs 4 into the adjacent filling chamber 1. The fluid connection 5 by channels is depicted schematically by the white arrows for the three lowermost filling chambers 1.

A chamber forming a flow channel 2 adjoins each filling chamber 1 on the right-hand side. In FIG. 1, only the flow channels 2 in the lowermost row and in the central row are denoted by the reference sign 2. Otherwise, the flow channels in FIG. 1 are depicted by black arrows pointing to the right. Each flow channel 2 is adjoined on the right-hand side by a receptacle chamber 3. Then, alternately, each receptacle chamber 3 is followed by a flow channel 2 and this in turn is followed by a receptacle chamber 3. Each receptacle chamber 3 is respectively connected to two reservoirs 4, analogously to the filling chambers 1. Each reservoir 4 in turn consists of a honeycomb of the honeycomb structure of the analysis plate 100. For the four lowermost rows of the receptacle chambers 3, the fluid connections 5 of the reservoirs to the adjacent receptacle chambers 3 are depicted as white arrows. The reference signs 5 can only be identified in the lowermost row of the reservoirs 4 in FIG. 1 and in the magnified illustration in FIG. 1a. Overall, only an L-shaped cutout of the honeycomb structure is depicted. The structure continues at least so far that the matrix of receptacle chambers 3 forms a complete square of eight by eight receptacle chambers 3. However, it is also possible to form larger structures, depending on necessity and usage purpose.

Further, it can be seen in FIG. 1 that the analysis plate 100 has perforations 101 on at least one lateral edge, which perforations serve to transport the analysis plate 100 by means of a suitable transportation device.

In the magnified illustration of FIG. 1a, it can be seen that each receptacle chamber 3 is connected to two flow channels 2, through which the liquid to be examined flows in and out, and that, furthermore, each receptacle chamber 3 is connected to two reservoirs 4, from which reagents can be filled into the receptacle chamber.

The filling chamber 1 and the flow channels 2 and the receptacle chambers 3 have a clear space of less than 1 mm between the walls parallel to one another.

The edge of the analysis plate 100 lying at the bottom in FIG. 1 is provided with perforations 101, similar to the perforations for transporting a film. Corresponding perforations are preferably arranged at the upper edge of the analysis plate. By means of the perforations, the analysis plate 100 can be transported into predefined positions by toothed wheels.

FIG. 2 depicts the complete analysis plate 100 with eight receptacle chambers 3 arranged next to one another. It can be seen that the flow channels 2 and the receptacle chambers 3 are covered by a cover layer 102. Thus, the analysis plate 100 of the analysis device is closed off on one side. The cover layer can be bonded onto the honeycomb structure of the analysis plate 100, after the filling chambers 1, flow channels 2, receptacle chambers 3 and reservoirs 4 and also fluid connections 5 were etched therein.

FIG. 3 schematically shows the filling process of the filling chambers 1. A filling piston 6 is held in a filling cylinder 105. The filling cylinder 105 is filled with a liquid 7. An outlet opening, which is provided with a micro barrier 8 for the liquid, is provided on the filling cylinder 105, lying opposite to the filling piston 6 and above the filling chamber 1 of the analysis plate 100.

The micro barrier 8 has a narrow passage channel. The passage channel has a water-repellent (hydrophobic) surface.

If the passage opening of the micro barrier 8 is situated above a filling chamber 1, the filling piston 6 is lowered by a defined path. As a result of this, a set amount of liquid 9 emerges and is filled into the filling chamber 1 of the analysis plate 100.

A store 35 for sealing plugs 10 can be seen on the left-hand side of the filling cylinder 105. A separating blade 36 is arranged under the store 35. After filling in the set amount of liquid 9 into the filling chamber 1, the separating blade 36 is actuated such that the edge thereof separates a sealing plug 10 from the number of sealing plugs 10 in the store 36.

The analysis plate 100 with the filling chambers 1 is then moved relative to the filling cylinder 105 until the micro barrier 8 is situated above the next filling chamber 1 (see FIGS. 3b-d). In the process, the rear section of the separating blade 36 presses the sealing plug into the already filled filling chamber 1. The filling chamber 1 is sealed as a result of this.

The aforementioned next filling chamber 1 is then filled by lowering the filling piston 6. During the onward transport of the analysis plate 100 with the filling chamber 1, the filled amount of liquid 9 is sheared off the liquid 7 in the filling cylinder 105 (see FIG. 3e). As long as no pressure is exerted onto the liquid 7 in the filling cylinder 105 no liquid remains in the passage channel of the micro barrier 8 as a result of the hydrophobic coating.

After filling the filling chamber 1, the amount of liquid 9 is led from the filling chamber 1 into the receptacle chambers 3. This process can be seen in the illustrations of FIG. 4. On the bottom, the filling chamber 1 is completed by a movable element, namely a filling piston 11. In FIG. 4a), the filling piston 11 is situated in the lower position. The filling piston 11 is pushed into the filling chamber 1. In the process, the filling piston 11 presses the amount of liquid 9 from the filling chamber 1 into the adjacent flow channel 2 (FIG. 4b). From there, the liquid flows into the adjacent receptacle chamber 8. The amount of liquid 9 then continues to flow into the subsequent flow channels 2 and receptacle chambers 3.

FIG. 4b) depicts the state when the filling chamber 1 is completely empty. It can be seen that the amount of liquid 9 only fills approximately half of the filling chambers 3 and flow channels 2.

In FIG. 4, it can be seen that a piston chamber 12 containing a separating piston 13 is arranged below each flow channel 2. The separating piston 13 forms a movable element for the onward transport of the liquid in the flow channel 2. It should be noted that merely the reference signs for the flow channels 2 and the receptacle chambers 3 are depicted in FIG. 4a), whereas FIG. 4f) shows the reference signs for the separating pistons 13 and the sealing pistons 13. For reasons of clarity, only the extreme right-hand piston chamber 12 is provided with a reference sign in FIG. 4f).

The piston chamber 12 and the separating piston 13 can both likewise be etched out of the glass material of the analysis plate 100. By pressing the first, extreme left-hand separating piston 13 against the ceiling of the first flow channel 2, the liquid is pressed out of the flow channel 2. This state is depicted for the flow channel 2 furthest left in FIG. 4c). In order to press out the liquid, a plunger 19 is pressed from below against the separating piston 13. If the second separating piston 13 is pressed against the ceiling of the flow channel 2 assigned thereto, the liquid is also driven out of this second flow channel (FIG. 4d). A small amount of the liquid is then enclosed in the receptacle chamber 3 between the two separating pistons 13. When this extreme left-hand receptacle chamber 3 is sealed, a plunger 19 is moved against the sealing piston 14 which delimits this receptacle chamber. The plunger 19 defines a predefined volume in the receptacle chamber 3, and so, when the receptacle chamber 3 is sealed by the separating piston 13 situated to the right therefrom, a certain liquid volume remains in the receptacle chamber 3.

Successively, all separating pistons 13 are pressed against the ceilings of the flow channels 2 assigned thereto by the plungers 19 lying therebelow (FIGS. 4e-4j), with the sealing piston 14 respectively lying to the left of the separating piston 13 being locked in the predefined position by the plunger 19 assigned thereto. After this process is complete, the amount of liquid 9 from the filling chamber 1 is split uniformly between the receptacle chambers 3 (see FIG. 4j).

In the lower region, each receptacle chamber 3 of the analysis plate 100 is sealed by a sealing piston 14. The sealing piston 14 is arranged displaceably in the receptacle chamber 3. It allows the volume of the receptacle chamber 3 to be changed. This ensures, firstly, that the receptacle chamber 3 is sealed against leakage of liquid but, secondly, that it has a changeable volume such that the volume of the receptacle chamber 3 corresponds to the volume of the filled liquid at all times during the filling process and when the temperature changes.

FIG. 5 shows three magnified illustrations of a plurality of flow channels 2 and adjacent receptacle chambers 3. It can be seen that the separating pistons 13 each have an edge 15 on their top side. During the axial displacement of the separating piston 13, the edge 15 is moved upward into an edge receptacle 16 in the region of the cover layer 102. The edge 15 separates the liquid film in the flow channel 2. The surface of the separating piston 13 and the cover wall of the flow channel 2 preferably have a hydrophobic embodiment. To this end, it can be either manufactured with a suitable surface roughness or coated to be hydrophobic.

Furthermore, it can be seen in FIGS. 5a-5c that each separating piston 13 has lateral latches 17, which are attached to the separating piston 13 in a resilient manner and latch into complementary latch receptacles (not depicted here) of the analysis plate 100 in the region of the wall of the piston chambers 12. As a result of this, each separating piston 13 is locked into its locking position, in which it rests against the upper wall of the flow channel 2. Further, it can be seen in these figures that the curvature of the top side of the separating piston 13 is greater than the curvature of the ceiling of the flow channel 2. In other words, when the separating piston 13 is pushed against the ceiling of the flow channel 2, a gap widening outward from the center is created. This gap prevents amounts of liquid from being enclosed between the ceiling of the flow channel 2 and the top side of the separating piston 13 when the flow channel 2 is sealed.

On their circumference, the sealing pistons 14 have cooling channels 18 (see FIG. 5), through which a cooling medium, in particular cooling air, can be guided. These cooling channels 18 serve to cool the liquid drop 21 in the receptacle chamber 3 so that the liquid follows a predefined temperature profile, in which a cooling phase can follow a heating phase, as exactly as possible. The cooling channels open into the receptacle chamber 3 itself such that cooling air can directly flow into the receptacle chamber 3 and flow out therefrom again. Here, the cooling channels 18 cooperate with further air-permeable micro-channels, as will be explained in more detail below in conjunction with FIGS. 8 and 9.

FIG. 6 shows the means for heating the liquid in the receptacle chambers 3. Plungers 19, which can be lifted by a micro linear drive, are arranged below the sealing pistons 14 for the receptacle chambers 3. Each plunger 19 is heated to a predefined temperature value. If a plunger 19 is lifted by a micro linear drive, it contacts the piston base of the sealing piston 14 and, via this piston base, transmits its temperature to the liquid drop 21 in the corresponding receptacle chamber 3. The different plungers 19 can have different temperature values so that different temperatures can successively be impressed onto the liquid in each receptacle chamber 3. The analysis plate 100 is transported over a matrix of plungers 19. Here, the transportation direction is denoted by the arrow 20. After each step-wise advance of the analysis plate, a different plunger 19 with a different temperature value can be pressed against the sealing piston 14 of a specific receptacle chamber 3. Predefined temperature cycles are realized in this manner.

Reference signs have to a large extent only been plotted in the upper FIG. 6a) for reasons of clarity.

Heating by means of plungers is depicted once again in FIG. 7 in a magnified fashion. The left-hand plunger 19 is pressed against the base of the sealing piston 14 and transfers the temperature to the liquid drop 21 in the receptacle chamber 3. The right-hand plunger 19 is withdrawn to the bottom, and so the liquid drop 21 in the right-hand receptacle chamber 3 is not heated.

FIG. 7 furthermore shows an arrangement for producing and recording optical signals, in particular fluorescence signals, in the liquid drops 21. An aperture plate 23, into which pinhole apertures 24 have been worked, is situated above the cover layer 102 of the analysis plate 100. The pinhole apertures 24 are arranged in such a way that respectively one pinhole aperture 24 is aligned with respect to one receptacle chamber 3. An optical sensor 25, e.g. a CMOS image sensor or a CCD image sensor, is situated above each pinhole aperture 24. By way of example, the basic area of each image sensor can be 1×1 mm. The pinhole of the pinhole aperture 24 is surrounded by a light source 26 on the side facing the receptacle, which light source preferably consists of light-emitting diodes (LEDs), in particular organic LEDs. By means of the LEDs, light can be radiated into the liquid drop 21 in each receptacle chamber 3. Subsequently, the fluorescent light emissions of the liquid drop 21 in each receptacle chamber 3 can be recorded by the optical sensor 25.

FIGS. 8 and 9 show a cooling device for the liquid drops 21 in the receptacle chambers 3. Cooling channels 27, through which air can be blown, are arranged in the upper cover layer 102 of the analysis plate 100. In practice, a cooling element, for example a Peltier element, can be arranged above the cooling channels 27. A pressure gradient ensuring that air flows through the analysis plate 100 can be produced by a fan or by a suction device situated below the analysis plate 100. The cooling channels 18 of the sealing pistons 14 and further cooling channels in the separating pistons 13 and the walls of the piston chambers 12 guide the airflow through the receptacle chambers 3 to the underside of the analysis plate 100. The ceiling of the receptacle chambers 3 and the piston walls, of the sealing piston 14, facing the liquid drop 21 in the receptacle chamber 3 are preferably hydrophilic and consequently well wet by the liquid drop 21 in the receptacle chamber 3. The sidewalls of the receptacle chambers 3 preferably have a hydrophobic embodiment such that they repel the liquid drop 21 in the receptacle chamber 3. The surface tension of the liquid drop in the receptacle chamber 3 and the very small cross sections of the cooling channels prevent the liquid from being dragged along by the cooling air and leaking through the cooling channels.

It can be seen that the cooling airflow also flows around the plungers 19. This ensures that a relevant heat transfer is not brought about by convection, but only by contact of the end face of a plunger 19 with the base wall of the receptacle chamber 3 formed by the piston base of the sealing piston 14. As soon as the plunger 19 is withdrawn and is no longer in contact with the piston base, the cooling airflow around the plunger 19 prevents further heat transfer from the plunger 19 to the sealing piston 14.

FIGS. 10 and 10a) show a micro linear drive 29 for the plungers 19. A ring-shaped element 30 has two angled slipways 31. A piezoelectric actuator 32 causes a rotation of the ring-shaped element 30 and, by means of the slipway 31, a lifting of the plunger 19 assigned thereto. The movement of the piezoelectric actuator 32 can be transferred to the plungers 19 in a different manner, e.g. hydraulically.

FIG. 11a shows a housing 40 of an analysis device with a bearing surface 39, over which the analysis plate is moved. The bearing surface 39 has a regular matrix of bores 37, through which plungers 19, lying therebelow, protrude. FIG. 11b shows the housing 40 without bearing surface such that the matrix of plungers 19 can be seen schematically, which plungers are respectively coupled to such a micro linear drive.

FIG. 12 shows a transportation device 33 for an analysis plate 100 with receptacle chambers 3. The transportation device 33 has a plurality of transportation toothed wheels 34, which mesh with the perforated edge regions (FIG. 1) of the analysis plate 100 and exactly position the analysis plate 100. The distance between two transportation toothed wheels 34 in the transportation direction is less than the length of the analysis plate 100. Pressure rollers 38, which press the analysis plate 100 against the bearing surface, are provided above the transportation toothed wheels 34.

Essential aspects of the development described herein will be listed again below, ordered according to numbers.

1. A method for analyzing a liquid, in which the liquid is split between a plurality of containers, characterized in that the liquid is fed to a transparent analysis plate (100) which has a matrix with a plurality of receptacle chambers (3), which are arranged adjacent to one another and closed on at least one side, wherein the liquid is split between the receptacle chambers (3).
2. The method of number 1, characterized in that the liquid flows through flow channels (2) leading to the receptacle chambers (3).
3. The method of number 1 or 2, characterized in that the receptacle chambers (3) and/or the flow channels (2) are covered by a continuous transparent cover layer (102).
4. The method of one of the preceding numbers, characterized in that the receptacle chambers (3) and/or the flow channels (2) are delimited by movable elements, wherein the volume of the receptacle chambers (3) and/or of the flow channel (2) is modified by moving the elements.
5. The method of number 4, characterized in that the movable elements are filling pistons (11), sealing pistons (14) or separating pistons (13).
6. The method of number 4 or 5, characterized in that the movable elements are transparent.
7. The method of one of the preceding numbers, characterized in that the liquid is filled into a filling chamber (1) in the transparent analysis plate (100) and guided from the filling chamber (1) into a flow channel (2), which guides the liquid into at least one receptacle chamber.
8. The method of number 7, characterized in that the filling chamber (1) has an upper opening which is sealed by means of a sealing plug (10) after filling in the liquid.
9. The method of one of the preceding numbers, characterized in that axially displaceable plungers (19) can be moved against a wall of a receptacle chamber (3).
10. The method of number 9, characterized in that each plunger (19) is coupled to an electronically actuated micro linear drive (29).
11. The method of number 10, characterized in that the micro linear drive (29) has an actuator or a part connected therewith, which runs on a slipway and converts movements of the actuator into vertical lifting or lowering movements.
12. The method of one of numbers 9 to 11, characterized in that the axially displaceable plungers (19) can be heated to a predefined temperature value.
13. The method of one of numbers 9 to 12, characterized in that each receptacle chamber (3) is delimited by an axially displaceable sealing piston (14), with a plunger (19) having a predefined temperature value being moved against the piston base, facing away from the liquid, of said piston.
14. The method of one of numbers 7 to 13, characterized in that an axially displaceable separating piston (13) is pressed against the ceiling of the flow channel (2) and separates the liquid film in the flow channel (2).
15. The method of number 14, characterized in that the separating piston (13) is displaceable in a piston chamber (12), wherein separating piston (13) and piston chamber (12) have complementary latches (17) and latch receptacles, which latch into one another when the separating piston (13) lies against the ceiling of the flow channel (2).
16. The method of number 14 or 15, characterized in that an edge (15) is provided on the top side of the separating piston (13), which edge is pressed against the ceiling of the flow channel (2) in order to separate the liquid film.
17. The method of number 16, characterized in that the edge (15) is arranged displaceably in the separating piston (13) and inserted into the separating piston (13) when pushing the separating piston (13) against the ceiling of the flow channel (2).
18. The method of one of the preceding numbers, characterized in that the receptacle chambers (3) can be provided with a hydrophilic surface, at least in the region of the surface of the sealing piston (14) and the ceiling of the receptacle chamber (3) lying opposite this surface.
19. The method of number 18, characterized in that the sidewalls of the receptacle chambers (3) and/or the walls of the flow channels (2) are provided with a hydrophobic surface.
20. The method of one of the preceding numbers, characterized in that reagents, in particular enzymes, from reservoirs (4) of the analysis plate (100) are filled into at least one of the following chambers:
filling chambers (1);
receptacle chambers (3).
   21. The method of number 20, characterized in that an ejection piston is moved into the reservoirs (4) in order to fill the reagents into the aforementioned chamber.
   22. The method of one of the preceding numbers, characterized in that cooling channels (18, 27) through which a coolant flows are provided in the analysis plate (100).
   23. The method of number 22, characterized in that at least one of the following components is provided with cooling channels (18, 27):
the cover layer (102);
the wall of the piston chamber (12);
the separating piston (13);
the sealing piston (14).
   24. A method for analyzing a liquid, in which the liquid is split between a plurality of containers, characterized by the following steps:
A) the liquid is fed to a transparent analysis plate, which has a matrix with a plurality of receptacle chambers (3), which are arranged adjacent to one another, wherein the liquid is split between the receptacle chambers (3),
B) a first temperature is impressed onto each receptacle chamber (3) in the analysis plate (100) through a wall of the receptacle chamber (3) during a first period of time,
C) a second temperature is impressed onto each receptacle chamber (3) in the analysis plate (100) through a wall of the receptacle chamber (3) during a second period of time.
   25. The method of number 24, characterized in that step C) is repeated such that predefined temperatures are successively impressed consecutively in each receptacle chamber (3).
   26. The method of number 24, characterized in that the temperature is impressed by means of a plunger (19) heated to a predefined temperature, wherein the analysis plate (100) is preferably transported by a transportation device from a first position to a second position and a receptacle chamber (3) is contacted by a first plunger (19) in the first position and contacted by a second plunger (19) in the second position.
   27. The method of one of numbers 24 to 26, characterized in that the analysis plate (100) is fed to at least one row of optical sensors (25) such that each optical sensor (25) is assigned to one of the receptacle chambers (3), wherein the optical sensors (25) are spaced apart by the distance corresponding to that between receptacle chambers (3) in a row of receptacle chambers (3).
   28. The method of number 27, characterized in that the optical sensor (25) is arranged above the receptacle chamber (3).
   29. The method of number 27 or 28, characterized in that the analysis plate (100) is fed to at least one row of light sources (26) such that each light source (26) is assigned to one of the receptacle chambers (3), wherein the light sources (26) are spaced apart by the distance corresponding to that between two receptacle chambers (3) in a row of receptacle chambers (3).
   30. The method of number 29, characterized in that the light source (26) is arranged above the receptacle chamber (3).
   31. The method of number 29 or 30, characterized in that in each case at least one light source (26) is arranged in the direct vicinity of an optical sensor (25).
   32. A device for analyzing a liquid, comprising at least one analysis plate (100) with a plurality of containers into which the liquid can be split, characterized in that the analysis plate (100) is transparent, in that the analysis plate (100) has a matrix with a plurality of receptacle chambers (3) arranged adjacent to one another and in that the analysis plate (100) has at least one movable element with which the liquid is split between the receptacle chambers (3).
   33. The device of number 32, characterized in that the analysis plate (100) has flow channels (2) leading to the receptacle chambers (3).
   34. The device of number 32 or 33, characterized in that the receptacle chambers (3) and/or the flow channels (2) of the analysis plate (100) are covered by a continuous and transparent cover layer (102).
   35. The device of one of numbers 32 to 34, characterized in that the analysis plate (100) has displaceable pistons (13, 14) which delimit the receptacle chambers (3) and/or the flow channels (2).
   36. The device of number 35, characterized in that the pistons (13, 14) consist of a transparent material.
   37. The device of one of numbers 32 to 36, characterized in that the analysis plate (100) has at least one filling chamber (1), from which a flow channel (2) leads into at least one receptacle chamber (3).
   38. The device of number 37, characterized in that the filling chamber (1) has an upper opening which can be sealed by a sealing plug (10) after filling in the liquid.
   39. The device of one of numbers 32 to 38, characterized by axially displaceable plungers (19) which can be moved against a wall of a receptacle chamber (3).
   40. The device of number 39, characterized in that each plunger (19) is coupled to an electronically actuatable micro linear drive (29).
   41. The device of number 40, characterized in that the micro linear drive (29) has an actuator or a part connected therewith, which runs on a slipway and converts movements of the actuator into vertical lifting or lowering movements.
   42. The device of one of numbers 39 to 41, characterized in that the axially displaceable plungers (19) are coupled to at least one heating device, which heats the plungers (19) to a predefined temperature value.
   43. The device of one of numbers 39 to 42, characterized in that each receptacle chamber (3) is delimited by an axially displaceable sealing piston (14), with a plunger (19) with a predefined temperature value being able to be moved against the piston base, facing away from the liquid, of said piston.
   44. The device of one of numbers 35 to 43, characterized by an axially displaceable separating piston (13) which can be moved against the ceiling of the flow channel (2).
   45. The device of number 44, characterized in that the top side of the separating piston (13) and the ceiling of the flow channel (2) have a hydrophobic surface.
   46. The device of number 44 or 45, characterized in that the separating piston (13) is displaceable in a piston chamber (12), wherein separating piston (13) and piston chamber (12) have complementary latches (17) and latch receptacles which latch into one another.
   47. The device of number 44, 45 or 46, characterized in that an edge (15) is provided on the top side of the separating piston (13), which edge can be pressed against the ceiling of the flow channel (2) for separating the liquid film.
   48. The device of one of numbers 44 to 47, characterized in that the edge (15) is displaceably arranged in the separating piston (13).
   49. The device of one of numbers 32 to 48, characterized by reservoirs (4) in the analysis plate (100), into which reagents, in particular enzymes, can be filled and which have a fluid connection to at least one of the following chambers:
filling chambers (1);
receptacle chambers (3).
   50. The device of number 49, characterized in that ejection pistons for filling the reagents into the aforementioned chamber are arranged in the reservoirs (4).
   51. The device of one of numbers 32 to 49, characterized in that the analysis plate (100) is provided with cooling channels (18, 27).
   52. The device of number 50, characterized in that at least one of the following components has cooling channels (18, 27):
the cover layer (102);
the wall of the piston chamber (12);
the separating piston (13);
the sealing piston (14).
   53. A device for analyzing a liquid, in particular of one of numbers 32 to 52, in which the liquid is split between a plurality of containers, characterized by the following features:
A) a transparent analysis plate (100), which has a matrix with a plurality of rows of receptacle chambers (3), arranged next to one another, for receiving a specific volume of the liquid,
B) a first row of axially displaceable plungers (19), the spacing of which corresponds to the spacing of the receptacle chambers (3), which plungers are heated by means of a heating device to a predefined temperature.
C) a transportation device, which moves the analysis plate (100) to a first position, in which the row of axially displaceable plungers (19) is opposite to a row of receptacle chambers (3) and each plunger (19) in the row can respectively be moved against a wall of a receptacle chamber (3), wherein the temperature of the plunger (19) is impressed upon the liquid in the receptacle chamber (3).
   54. The device of number 53, characterized in that the transportation device is embodied to transport the analysis plate (100) to further positions, in which other axially displaceable plungers (19) with other temperatures can be moved against the wall of the receptacle chamber (3).
   55. The device of number 54, characterized in that the transportation device is embodied to move the analysis plate (100) back into the first position.
   56. The device of one of numbers 53 to 55, characterized in that it has at least one row of optical sensors (25), the spacing between one another corresponds to the spacing between receptacle chambers (3) in a row of receptacle chambers (3) in the analysis plate (100).
   57. The device of number 56, characterized in that the optical sensor (25) is arranged above the receptacle chamber (3).
   58. The device of number 56 or 57, characterized in that it has at least one row of light sources (26), the spacing between one another corresponds to the spacing between receptacle chambers (3) in a row of receptacle chambers (3) in the analysis plate (100).
   59. The device of number 58, characterized in that the light source (26) is arranged above the receptacle chamber (3).
   60. The method of number 58 or 59, characterized in that in each case at least one light source (26) is arranged in the direct vicinity of an optical sensor (25).
   61. A method for producing an analysis plate (100), in particular for a device of one of numbers 32 to 60, characterized in that a transparent plate, in particular made of glass, is irradiated by a focused laser beam such that the transparent material can be removed by etching in the focus volume.
   62. The method of number 60, characterized in that, by moving the focus with the aid of a laser scanner, the focus volume is moved through the transparent analysis plate (100) such that at least one of the following structures is embodied:
a plurality of receptacle chambers (3) arranged adjacent to one another;
flow channels (2), which interconnect a plurality of receptacle chambers (3);
displaceable separating pistons (13) or sealing pistons (14), which delimit the receptacle chambers (3) and/or flow channels (2);
at least one filling chamber (1);
a piston chamber (12);
complementary latches (17) and latch receptacles, which latch with one another, on separating pistons (13) and piston chamber (12);
an edge (15), on the top side of the separating piston (13), which is preferably displaceable;
cooling channels (18, 27).
   63. The method of number 61 or 62, characterized in that different surface roughnesses are produced, wherein, in particular, smooth hydrophilic surfaces and rough hydrophobic surfaces are produced.
   64. The method of number 63, characterized in that a hydrophobic surface with high surface roughness is produced by selective laser etching.
   65. The method of number 64, characterized in that a hydrophilic surface is produced by smoothing a hydrophobic surface by means of a laser beam.

### LIST OF REFERENCE SIGNS

1 Filling chamber
2 Flow channel
3 Receptacle chamber
4 Reservoir
5 Fluid connection
6 Filling piston
7 Liquid
8 Micro barrier
9 Amount of liquid
10 Sealing plug
11 Movable element, filling piston
12 Piston chamber
13 Movable element, separating piston
14 Sealing piston
15 Edges
16 Edge receptacle
17 Latch
18 Cooling channel
19 Plunger
20 Transportation direction
21 Liquid drop
22 Laser diodes
23 Aperture plate
24 Pinhole aperture
25 Optical sensor
26 Light source, LED
27 Cooling channel
28 Peltier element
29 Micro linear drive
30 Ring-shaped element
31 Slipway
32 Piezoelectric actuator
33 Transportation device
34 Transportation toothed wheel
35 Store
36 Separating blade
37 Bore
38 Pressure roller
39 Bearing surface
40 Housing
100 Analysis plate
101 Perforation
102 Cover layer
105 Filling cylinder

1-16. (canceled)

17. A method for analyzing a liquid, comprising:
feeding the liquid to a transparent analysis plate which has a matrix with a plurality of receptacle chambers which are arranged adjacent to one another and closed on at least one side, wherein the liquid is split between the receptacle chambers;
moving axially-displaceable plungers against walls of the receptacle chambers; and
transporting the analysis plate to the plungers using a transportation device.

18. The method as claimed in claim 17, wherein at least one of the plungers is coupled to a micro-linear drive that moves the at least one plunger.

19. The method as claimed in claim 17, wherein the liquid is split between the receptacle chambers using movable elements of the analysis plate, and wherein at least one of the plungers is moved against a movable element.

20. The method as claimed in claim 17, wherein at least one of the plungers is heated to a predefined temperature value.

21. The method as claimed in claim 17, wherein the liquid flows through flow channels leading to the receptacle chambers.

22. The method as claimed in claim 17, wherein the receptacle chambers and/or the flow channels are delimited by movable elements movable using at least one of the plungers, wherein a volume of the receptacle chambers and/or of the flow channel is modified by moving the movable elements.

23. The method as claimed in claim 22, wherein the movable elements are filling pistons, sealing pistons or separating pistons.

24. The method as claimed in claim 17, wherein the liquid is filled into a filling chamber in the transparent analysis plate and guided from the filling chamber into a flow channel, which guides the liquid into at least one receptacle chamber.

25. The method as claimed in claim 24, wherein least one of the following features is provided:
(i) the filling chamber has an upper opening, which is sealed by a sealing plug after filling in the liquid; or
(ii) the liquid is pressed out of the filling chamber by filling pistons.

26. The method as claimed in claim 17, wherein each of the receptacle chambers is delimited by an axially-displaceable sealing piston, with at least one of the plungers being moved against the piston base, facing away from the liquid, of said piston.

27. The method as claimed in claim 21, wherein an axially displaceable separating piston is pressed against the ceiling of at least one of the flow channels using at least one of the plungers and separates the liquid film in the at least one flow channel.

28. The method as claimed in claim 17, wherein reagents from reservoirs of the analysis plate are filled into at least one of the following chambers: filling chambers or receptacle chambers.

29. The method as claimed in claim 28, wherein the reagents are enzymes.

30. The method as claimed in claim 28, wherein at least one of the plungers is used to move an ejection piston in the reservoirs in order to fill the reagents into the aforementioned chamber.

31. A device for analyzing a liquid, comprising:
at least one analysis plate with a plurality of receptacle chambers into which the liquid can be split, wherein the analysis plate is transparent and wherein the analysis plate has a matrix with the plurality of receptacle chambers arranged adjacent to one another; and
plungers which are axially displaceable against walls of the receptacle chambers; and
a transportation device that transports the analysis plate to the plungers.

32. The device as claimed in claim 31, wherein the analysis plate has at least one movable element, wherein the movable element splits the liquid between the receptacle chambers, and wherein at least one displaceable plunger is movable against the movable element.

33. The device as claimed in claim 31, wherein the analysis plate has flow channels which lead to the receptacle chambers.

34. The device as claimed in claim 31, wherein the receptacle chambers and/or the flow channels of the analysis plate are covered by a continuous transparent cover layer.

35. The device as claimed in claim 31, wherein the analysis plate has displaceable pistons, which delimit the receptacle chambers and/or the flow channels.

36. The device as claimed in claim 31, wherein the analysis plate has at least one filling chamber, from which a flow channel leads into at least one of the receptacle chambers.

37. The device as claimed in claim 17, further comprising:
reservoirs in the analysis plate into which reagents can be filled and which have a fluid connection to at least one of the following chambers: filling chambers or the receptacle chambers.

38. The device as claimed in claim 37, further comprising:
an ejection piston is moved into the reservoirs in order to fill the reagents into the filling chambers of the receptacle chambers.
